(19) United States
(12) Patent Application Publication
(10) Pub. No.: US 2014/0128631 A1
(43) Pub. Date: May 8, 2014

(54) PROCESS FOR THE PREPARATION OF ALKANOIC ACID ESTERS IN A CARBONYLATION PROCESS USING PALLADIUM BIDENTATE BIPHOSPHATE LIGANDS

(71) Applicants: Johannes Gerardus De Vries, Echt (NL); Natascha Sereinig, Echt (NL); Elisabeth Wilhelmina Maria Van De Vondervoort, Echt (NL); Michele Catherine Christianne Janssen, Echt (NL)

(72) Inventors: Johannes Gerardus De Vries, Echt (NL); Natascha Sereinig, Echt (NL); Elisabeth Wilhelmina Maria Van De Vondervoort, Echt (NL); Michele Catherine Christianne Janssen, Echt (NL)

(73) Assignee: DSM IP ASSETS B.V., Heerlen (NL)

(21) Appl. No.: 14/008,683

(22) PCT Filed: Mar. 30, 2012
(86) PCT No.: PCT/EP12/55768

§ 371 Date: Nov. 13, 2013

Related U.S. Application Data
(60) Provisional application No. 61/470,673, filed on Apr. 1, 2011.

(30) Foreign Application Priority Data
Apr. 1, 2011 (EP) 11160912.9

(51) Int. Cl. C07C 67/36

(52) U.S. Cl. CPC C07C 67/36
USPC 560/204

(57) ABSTRACT

The invention relates to a carbonylation process for the preparation of an alkanoic acid ester comprising reacting:
(a) an alkene;
(b) a source of Pd;
(c) a bidentate phosphine ligand of formula I;

R¹R²P—R³—R—R⁴—PR⁵R⁶ (I)

wherein P represents a phosphorus atom; R1, R2, R⁵ and R⁶ can independently represent the same or different optionally substituted organic groups containing a tertiary carbon atom through which the group is linked to the phosphorus atom; R³ and R⁴ independently represent optionally substituted lower alkylene groups and R represents an optionally substituted aromatic group;
(d) a source of anions derived from an acid with a pKa<3;
(e) carbon monoxide; and
(f) an alkanol;
   characterized in that the process is performed in the presence of between 0.1 and 3 wt water.

The process advantageously has a high conversion rate and is suitable for the production of dimethyl adipate, adipate and hexamethylene diamine and products derived thereof such as nylon 6,6 from renewable sources such as plant waste, sewage waste etceteras instead of using fossil sources.

### FIELD OF THE INVENTION

The present invention relates to a carbonylation process for the preparation of an alkanoic acid ester using a Pd bidentate biphosphate ligand. The invention also relates to the production of polymers based on adipic acid.

### BACKGROUND OF THE INVENTION

This invention relates to a carbonylation process for the preparation of an alkanoic acid ester, said process comprising reacting:
an alkene;
a source of Pd;
a bidentate di-phosphine ligand of formula I,

R¹R²>P—R³—R—R⁴—P<R⁵R⁶ (I)

wherein P represents a phosphorus atom; R¹, R², R⁵ and R⁶ can independently represent the same or different optionally substituted organic groups containing a tertiary carbon atom through which the group is linked to the phosphorus atom; R³ and R⁴ independently represent optionally substituted lower alkylene groups and R represents an optionally substituted aromatic group;
a source of anions derived from an acid with a pKa<3;
carbonmonoxide; and
an OH group comprising compound.

In WO2001068583 is described the use of a bidentate diphosphate ligand of formula I for the carbonylation of ethylenically unsaturated compounds such as methyl 3-pentenoate.

A disadvantage of said process is that the conversion rate is insufficient. Although it is possible to increase the rate of the reaction by adding more catalyst this higher concentration of palladium accelerates its deactivation in the form of palladium black, which precipitates from the reaction. It is also possible to increase the rate of the reaction by increasing the temperature, but this will generally lead to lower selectivities and it also has the unwanted side effect of accelerating the formation of palladium black. Thus, the problem to be solved was to increase the rate of the reaction without increasing the palladium concentration and without increasing the temperature.

### DETAILED DESCRIPTION OF THE INVENTION

In a first aspect, the invention relates to a carbonylation process for the preparation of an alkanoic acid ester comprising reacting:
(a) an alkene;
(b) a source of Pd;
(c) a bidentate phosphine ligand of formula I;

R¹R²P—R³—R—R⁴—PR⁵R⁶ (I)

wherein P represents a phosphorus atom; R1, R2, R⁵ and R⁶ can independently represent the same or different optionally substituted organic groups containing a tertiary carbon atom through which the group is linked to the phosphorus atom; R³ and R⁴ independently represent optionally substituted lower alkylene groups and R represents an optionally substituted aromatic group;
(d) a source of anions derived from an acid with a pKa<3;
(e) carbon monoxide; and
(f) an alkanol;
   under condition wherein an alkanoic acid ester is produced, characterized in that the process is performed in the presence of between 0.1 and 3% wt water.

Preferably, the lower alkylene groups which R³ and/or R⁴ represent are non-substituted. R³ and R⁴ may independently represent —CH₂— or —C₂H₄—. In a preferred embodiment R¹, R², R⁵, and R⁶ are tert-butyl, R³ and R⁴ are methylene, and R is ortho-phenylene.

Suitable sources of Pd in the process of the invention include its salts, such as for example the salts of palladium and halide acids, nitric acid, sulphuric acid or sulphonic acids; palladium complexes, e. g. with carbon monoxide, dienes, such as dibenyzlideneacetone (dba) or acetylacetonate, palladium nanoparticles or palladium combined with a solid carrier material such as carbon, silica or an ion exchanger. Preferably, a salt of palladium and a carboxylic acid is used, suitably a carboxylic acid with up to 12 carbon atoms, such as salts of acetic acid, proprionic acid, butanoic acid or 2-ethyl-hexanoic acid, or salts of substituted carboxylic acids such as trichloroacetic acid and trifluoroacetic acid. A very suitable source is palladium (II) acetate.

In a preferred embodiment the source of Pd is selected from the group consisting of palladium halide, palladium carboxylate or Pd2(dba)3.

The amount of palladium used in the process according to the first aspect of the invention is the result of careful optimisation in an iterative process known to someone skilled in the art. Whereas high palladium concentrations lead to very fast reactions, they may also result in the formation of palladium black. This latter deactivation process is ameliorated by the presence of ligands and the pentenoate esters. The palladium black formation is also accelerated by high temperatures. In general a range of 10⁻⁷ to 10⁻¹ gram atom per mole of alkene will be the starting point of this optimisation. More likely, the palladium amount will be in the range of 10⁻⁵ to 10⁻² gat per mole of alkene.

Vavasori et al. (Journal of Molecular Catalysis A: Chemical (2003), vol. 191, p. 9-21) describe that the conversion rate of the hydroesterification of cyclohexene using a Pd(PPh3)2(TsO)2 complex can be increased when the process is performed in the presence of water. However, according to Vavasori et al. the amount of water should not exceed 0.3% (3000 ppm) lest the Pd hydride decomposes to metallic Pd. However, the complex used by Vavasori et al. is not very stable and hence unsuitable for a large-scale alkoxycarbonylation process. Although much better and much more stable alkoxcycarbonylation catalysts are known and used in large-scale processes, the effect of water on these carbonylations has not been reported which makes it highly unlikely that it will be successful in these cases.

The inventors have surprisingly found that the conversion rate (TOF, h⁻¹) of a carbonylation process for the preparation of an alkanoic acid ester comprising reacting:
(a) an alkene;
(b) a source of Pd;
(c) a bidentate phosphine ligand of formula I;

R¹R²P—R³—R—R⁴—PR⁵R⁶ (I)

wherein P represents a phosphorus atom; R1, R2, R⁵ and R⁶ can independently represent the same or different optionally substituted organic groups containing a tertiary carbon atom through which the group is linked to the phosphorus atom; R³ and R⁴ independently represent optionally substituted lower alkylene groups and R represents an optionally substituted aromatic group;
(d) a source of anions derived from an acid with a pKa<3;
(e) carbon monoxide; and
(f) an alkanol may be increased when said process is performed in the presence of between 0.1-3% wt water.

Preferably the amount of water in the process of the first aspect of the invention is between 0.13 and 3% wt, more preferably between 0.19 and 3%, between 0.19 and 2.55% wt, even more preferably between 0.24 and 2.55% wt, even more preferably between 0.51 and 2.55% wt. Preferred lower limits of the amount of water are at least 0.2% wt, 0.25% wt, 0.3% wt, 0.35% wt, and 0.4% wt. Preferred upper limits of the amount of water are 0.6% wt, 0.7% wt, 0.8% wt, 0.9% wt, 1% wt, 1.1% wt, 1.2% wt, 1.3% wt, 1.4% wt, 1.5% wt, 1.6% wt. A preferred amount of water is between 0.15 and 1.5% wt.

In a preferred embodiment the alkanoic acid ester is an ester of formula II,

XOOC—(CH₂)₄—COOY (II)

wherein X and Y are independently a lower alkyl group and/or H.

The lower alkyl group preferably has 4 C atoms or less, more preferably 3 C atoms or less, even more preferably 2 C atoms or less, most preferably the lower alkylgroup is methyl.

In one embodiment the alkanoic acid ester of formula II is adipate monoester.

In another, highly preferred embodiment the alkanoic acid ester of formula II is adipate dimethylester. Adipate dimethyl ester is an important intermediate in the production of adipic acid (1,6-hexanedioic acid), which is an important precursor for inter alia the production of polyamides such as Nylon 6,6 or Stanyl™. Further, esters of adipic acid may be used in plasticisers, lubricants, solvent and in a variety of polyurethane resins. Other uses of adipic acid are as food acidulants, applications in adhesives, insecticides, tanning and dyeing. The alkanoic acid ester of formula II is understood to also include higher esters, e.g. tri, four, five, and even polyesters.

Suitable alkenes may comprise between 2 to 50 carbon atoms per molecule, or maybe mixtures of alkenes. They may be terminal or internal alkenes, they may be cis- or trans-alkenes. Suitable alkenes may have one or more isolated or conjugated unsaturated bonds per molecule. Preferred are alkenes having from 2 to 20 carbon atoms, or mixtures thereof. More preferred are alkenes having 18 carbon atoms or less, even more preferred 16 carbon atoms or less, or 10 carbon atoms or less. The alkene may comprise functional groups or heteroatoms, such as nitrogen, sulphur or oxygen. These functional groups or heteroatoms may be attached to the olefinic carbons or to the other carbons in the alkene. Examples include alcohols, aldehydes, carboxylic acids, esters or nitriles as functional groups. In a preferred embodiment, the alkene is 1,3-butadiene, ethene, propene, butene, isobutene, pentene, pentene nitrile, alkyl pentenoate such as cis and trans methyl 2-pentenoate, cis and trans methyl 3-pentenoate, methyl 4-pentenoate, pentenoic acid, such as cis and trans 2-, 3, and 4-pentenoic acid, heptene, vinyl esters such as vinyl acetate, octenes, dodecenes. If the alkene contains more than one olefinic group either one or all olefinic groups can be alkoxycarbonylated.

The alkene preferably comprises methyl 2-pentenoate. In a preferred embodiment the alkene is a pentenoate ester. Said pentenoate ester is preferably methyl pentenoate, more preferably a mixture comprising cis- and/or trans-methyl 2-pentenoate, cis- and/or trans-methyl 3-pentenoate, and/or methyl-4-pentenoate.

The most important process to produce adipic acid is based on oil and starts from benzene. In this process benzene is hydrogenated to cyclohexane. Cyclohexane is then oxidised using HNO₃ as oxidant to adipic acid. A disadvantage of this process is that it is based on fossil derived oil. Another disadvantage is the evolution of NOₓ during the oxidations step, which either is vented to the air, which is highly undesirable as it is a greenhouse gas, or is catalytically destroyed, which is an expensive process. New processes for the production of adipic acid have been developed based on butadiene, which is converted tot methyl 3-pentenoate. The next step is isomerisation of methyl 3-pentenoate to methyl 4-pentenoate which can be converted to dimethyladipate. A disadvantage of the butadiene-based processes is the high cost of butadiene. A second disadvantage is the low rate of the methoxycarbonylation of butadiene. Another process for the production of adipic acid starts from levulinic acid as a renewable source. Levulinic acid may be produced from agricultural waste products or waste from the paper industry or municipal waste and therefore constitutes a renewable source of a C-5 fragment. The hydrogenation of levulinic acid has been described and produces valerolactone in high yield. A number of patents exist describing the reaction of valerolactone with methanol, either in the liquid phase or in the gas phase to deliver a mixture of methyl 2-pentenoate, methyl 3-pentenoate and methyl 4-pentenoate.

The mixture comprising cis- and/or trans-methyl 2-pentenoate, cis- and/or trans-methyl 3-pentenoate, and/or methyl-4-pentenoate may comprise other components, such as free pentenoic acids (2-pentenoic acid, 3-pentenoic acid, and/or 4-pentenoic acid) and valerolactone. Preferably, the amount of methyl 2-pentenoate in said mixture is between 5-85 wt %.

In another embodiment the alkene is ethene. The product of the methoxycarbonylation of ethene, methyl proprionate can be further reacted with formaldehyde to form methyl methacrylate. Thus the present invention can lower they cost of an already existing process for the production of methyl methacrylate.

In an embodiment the OH group comprising compound is an alkanol, preferably methanol.

The process of the invention is optionally performed in the presence of an additional solvent. In practice, diester of adipic acid or the heavies that build up during the recycle of the catalyst may function as a solvent. The additional solvent is preferably an aprotic solvent. Suitable solvents include ketones, such as for example methylbutylketone; ethers, such as for example anisole (methyl phenyl ether), 2,5,8-trioxanonane (diglyme), diethylether, tetrahydrofuran, 2-methyl-tetrahydrofuran, diphenylether, diisopropylether and the dimethylether of di-ethyleneglycol; esters, such as for example ethyl acetate, methyl acetate, dimethyl adipate and butyrolactone; amides, such as for example dimethylacetamide and N-methylpyrrolidone; and sulfoxides and sulphones, such as for example dimethylsulphoxide, di-isopropylsulphone, sulfolane (tetrahydrothiophene-2,2-dioxide) 2-methylsulfolane and 2-methyl-4-ethylsulfolane. Very suitable are aprotic solvents having a dielectric constant that is below a value of 50, more preferably in the range of 3 to 8, at 298.15 K and 1 bar. If the hydroxyl group containing compound is an alkanol, a further preferred aprotic solvent is the ester carbonylation product of the alkene, carbon monoxide and the alkanol.

The molar ratio of bidentate phosphine of formula Ito palladium is from 1-10, preferable from 2-6.

Suitable reaction temperatures are in the range of 20-180° C., more preferably 20-160° C., even more preferably in the range of 50-120° C.

The pressure in the process of the invention is preferably between 5 and 100 bar, more preferably between 10 and 50 bar.

The source of anions derived from acid having a pKa below 3.0 (measured in aqueous solution at 18° C.) preferably is a non-coordinating anion. Hereby is meant that little or no covalent interaction takes place between the palladium and the anion.

Examples of suitable anions include anions of phosphoric acid, sulphuric acid, sulphonic acids and halogenated carboxylic acids such as trifluoroacetic acid.

Sulphonic acids are in particular preferred, for example trifluoromethanesulphonic acid, p-toluenesulphonic acid and 2,4,6-trimethylbenzene sulphonic acid, 2-hydroxypropane-2-sulphonic acid, tert-butyl sulphonic acid, methyl sulphonic acid. The acid can also be an ion exchange resin containing sulphonic acid groups.

An especially preferred source of anions derived from an acid having a pKa below 3.0 is methylsulphonic acid, tert-butyl sulphonic acid and/or 2,4,6-trimethylbenzenesulphonic acid.

The molar ratio of the source of anions and palladium is preferably between 1:1 and 100:1 and more preferably between 1:1 and 10:1.

Carbon monoxide partial pressures in the range of 1-100 bar are preferred. In the process according to the present invention, the carbon monoxide can be used in its pure form or diluted with an inert gas such as nitrogen, carbon dioxide or noble gases such as argon. Small amounts of hydrogen can also be present. In general, the presence of more than 5% hydrogen is undesirable, since this can cause hydroformylation or even hydrogenation of the pentenoate esters.

In a second aspect the invention provides a process to produce adipic acid dimethyl ester, said process comprising:
(a) converting valerolactone into methyl pentenoate by treatment with methanol, in the presence of an acidic or basic catalyst in the gas phase or in the liquid phase; and
(b) converting the methyl pentenoate produced in step (a) to adipic acid dimethyl ester in a carbonylation process according to the first aspect of the invention wherein the alkanol is methanol.

The inventor has surprisingly found that the process of the second aspect of the invention may be advantageously carried out without an additional step after step (a) and before step (b), such as a purification or separation step to remove or reduce the amount of methyl-2-pentenoic acid.

The conversion of valerolactone to a mixture of methyl pentenoates in step (a) can be done either in the liquid phase or in the gas phase to deliver a mixture of methyl 2-pentenoate, methyl 3-pentenoate and methyl 4-pentenoate. Such processes have been described in WO 2005058793, WO 2004007421, U.S. Pat. No. 4,740,613.

In an embodiment valerolacton is prepared by converting levulinic acid to valerolactone in a hydrogenation reaction. Such processes are for example described in L. E. Manzer, Appl. Catal. A, 2004, 272, 249-256; J. P. Lange, J. Z. Vestering and R. J. Haan, Chem. Commun., 2007, 3488-3490; R. A. Bourne, J. G. Stevens, J. Ke and M. Poliakoff, Chem. Commun., 2007, 4632-4634; H. S. Broadbent, G. C. Campbell, W. J. Bartley and J. H. Johnson, J. Org. Chem., 1959, 24, 1847-1854; R. V. Christian, H. D. Brown and R. M. Hixon, J. Am. Chem. Soc., 1947, 69, 1961-1963.;L. P. Kyrides and J. K. Craver, U.S. Pat. No. 2,368,366, 1945; H. A. Schuette and R. W. Thomas, J. Am. Chem. Soc., 1930, 52, 3010-3012.

In another embodiment levulinic acid is prepared by converting a C6 carbohydrate to levulinic acid in a hydrolysis reaction. Such processes are for example described in L. J. Carlson, U.S. Pat. No. 3,065,263, 1962; B. Girisuta, L. P. B. M. Janssen and H. J. Heeres, Chem. Eng. Res.Des., 2006, 84, 339-349; B. F. M. Kuster and H. S. Vanderbaan, Carbohydr. Res., 1977, 54,165-176; S. W. Fitzpatrick, WO8910362, 1989, to Biofine Incorporated; S. W. Fitzpatrick, WO9640609 1996, to Biofine Incorporated.. Examples of C6 carbohydrates are glucose, fructose, mannose and galactose. Preferred raw material for the C6 carbohydrates is lignocellulosic material containing carbohydrate based polymers composed partly or entirely from C6 sugars such as cellulose, starch and hemicellulose. The C6 carbohydrate may comprise other components, such as plant waste, paper waste, sewage etc.

The process to produce adipic acid according to the second aspect of the invention advantageously allows the use of renewable sources such as plant waste, sewage waste etceteras instead of using fossil sources.

In a preferred embodiment, the process according to the second aspect of the invention includes isolating dimethyl adipate, e.g. by distillation. Unconverted methyl pentenoates and/or catalyst containing distillation residue and which may still contain some dimethyl adipate may be recycled back into the reactor.

In an embodiment dimethyladipate is hydrolyzed to adipic acid in a hydrolysis reaction. The hydrolysis of DMA to adipic acid is well known to the person skilled in the art.

In another embodiment adipic acid is converted to ammonium adipate by treatment with ammonia.

In another embodiment ammonium adipate is converted to adiponitril in a dehydration reaction.

In another embodiment adiponitril is converted to hexamethylenediamine in a reduction reaction. The conversion of adipate to ammonium adipate, from ammonium adipate to adiponitril and from adiponitril to hexamethylene diamine is known to persons skilled in the art and is for example described by Fernelius et al. (Journal of Chemical Education, 1979, vol. 56, p. 654-656).

The following examples are for illustrative purposes only and are not to be construed as limiting the invention.

The following examples are for illustrative purposes only and are not to be construed as limiting the invention.

### EXAMPLES

### Examples 1-3

### Preparation of a Mixture of Methyl Pentenoates

The catalyst (Grace-Davison/Davicat SIAL 3501, 21.2 g) was loaded into a tubular gas phase reactor at atmospheric pressure and then heated to 255° C. The reaction temperature was monitored inside the reactor with a thermocouple. Prior to the introduction of the feed, the desired reaction temperature and pressure were achieved under flowing nitrogen. Gas flow to the reactor was controlled using Brooks mass flow controllers. Upon reaching the desired conditions, a solution of γ-valerolactone in MeOH (1:1 in weight) was prepared, preheated to 190° C. and fed to the packed-bed tubular reactor using a HPLC pump. The liquid effluent was collected for quantitative analysis in a separator at ambient temperature and analyzed by GC. The LHSV w.r.t. valerolactone was 0.49. Samples from three different runs were distilled. The composition of the main fraction from these three runs is listed below in Table 1. In all mixtures more than 5 mol % of methyl 2-pentenoate was present.

TABLE 1
Mass percentages methyl pentenoates in mixtures obtained from the gasphase
reaction between valerolactone and methanol

| | | | | | | | Valero- | | |
|---|---|---|---|---|---|---|---|---|---|
| | MeOH | c-M2P | M4P | tr-M3P | c-M3P | tr-M2P | Lactone | Water | Total |
| Example | m/m % | m/m % | m/m % | m/m % | m/m % | m/m % | m/m % | m/m % | m/m % |
| 1 | 1.1 | 2.2 | 25.0 | 44.6 | 16.3 | 8.7 | 1.1 | 1.1 | 100 |
| 2 | 0.0 | 0.8 | 18.3 | 48.3 | 17.5 | 11.7 | 0.8 | 0.8 | 98 |
| 3 | 0.0 | 1.6 | 12.9 | 48.4 | 17.7 | 14.5 | 1.6 | 1.6 | 98 |

### Examples 4

### Methoxycarbonylation of Methyl Pentenoates

A solution of α,α′-Bis(di-tert-butylphosphino)-o-xylene (20 μmol, from Strem Chemicals, Inc., 15, rue de l'Atome, Z.I., 67800 BISCHHEIM, France); 5 eq. in 5 mL methanol), was added to Pd precursor (4 μmol Pd(OAc)₂). Methanesulfonic acid (MSA, 4 μl, 40 μmol, 10 eq.) was added to the catalyst solution upon which the color changed from yellow to orange. Substrate (either methyl 2-pentenoate, methyl 3-pentenoate, or a mixture of methyl 2-pentenoate, methyl 3-pentenoate and methyl 4-pentenoate as obtained in Examples 1-3 or pre-maid in a 1:1:1 ratio) was added and the mixture was transferred into a glass insert of an Endeavour (set-up of 8 small autoclaves fitted with an overhead stirrer). The reactors were purged 5 times with N₂ and thereafter 10 times with 20 bar of CO. The reactors were pressurized to 20 bar and heated to the indicated reaction temperature. The reaction vessels were cooled down to room temperature after 1 h and the pressure was released. Conversion to dimethyl adipate and selectivities were determined by means of GC analysis (Table 2).

(M2P=methyl 2-pentenoate; M3P=methyl 3-pentenoate; M4P=methyl 4-pentenoate). Mixtures of M2P, M3P and M4P were obtained in a gas phase reaction by reaction between valerolactone and methanol, as described above)

TABLE 2
Methoxycarbonylation of methyl pentenoates

| | | | | | Selectivity to |
|---|---|---|---|---|---|
| | | | | | dimethyl |
| | Example | Substrate | Temp (° C.) | TOF (h⁻¹) | adipate (%) |
| Examples of | 4 | M2P | 50 | 105 | 100 |
| the process | 5 | M2P | 75 | 289 | 100 |
| according to | 6 | M2P | 100 | 417 | 97 |
| the invention | 7 | Mixture from Example 1 | 75 | 362 | 100 |
| | 8 | Mixture from Example 2 | 75 | 335 | 100 |
| | 9 | Mixture from Example 3 | 75 | 482 | 97 |
| | 10 | M2P, M3P, M4P (1:1:1) | 75 | 309 | 100 |
| | 11 | M2P, M3P, M4P (1:1:1) | 100 | 342 | 97 |
| Comparative | 12 | M3P | 50 | 141 | 100 |
| examples | 13 | M3P | 75 | 281 | 100 |
| | 14 | M3P | 100 | 332 | 98 |

Table 2 shows that methyl 2-pentenoate may be converted at practically the same rate and with the same selectivities as methyl 3-pentenoate at 50, 75 and 100° C. In addition, the mixture containing methyl 2-pentenoate, methyl 3-pentenoate and methyl 4-pentenoate was also converted to methyl adipate with the same rate and selectivity as methyl 3-pentenoate. This experiment shows that it is possible to use the mixture of methyl pentenoates obtained by converting valerolactone in the methoxycarbonylation reaction to dimethyl adipate and that the presence of methyl 2-pentenoate in this mixture has no adverse effects.

### Examples 13-19

### Effect of Added Water

A solution of α,α′-Bis(di-tert-butylphosphino)-o-xylene (40 μmol, 5 eq.) in 4 mL methanol was added to the Pd precursor (8 μmol Pd(OAc)₂). Methanesulfonic acid (8 μl, 80 μmol, 10 eq.) was added to the catalyst solution upon which a color change was visible from yellow to orange. Methyl 3-pentenoate and water (for amounts see Table 3) were added and the mixture was transferred into a glass Endeavor insert. The reactors were purged 5 times with N₂ and thereafter 10 times with 20 bar of CO. The reactors were pressurized to 20 bar with CO and heated to the indicated reaction temperature. The reaction vessels were cooled down to room temperature after 1 h and the pressure was released. Conversions and selectivities were determined by means of GC analysis. The exact amounts of water were determined by Karl Fisher titration. Results are shown in Table 3.

TABLE 3
Effect of water

| Example | Wt % water | C (%) | Sel to DMA (%) | TOF (h⁻¹) |
|---|---|---|---|---|
| 13 | 0.13 | 75 | 95 | 562 |
| 14 | 0.19 | 78 | 96 | 591 |
| 15 | 0.24 | 79 | 97 | 598 |
| 16 | 0.51 | 78 | 94 | 611 |
| 17 | 1.185 | 75 | 96 | 591 |
| 18 | 1.7 | 70 | 91 | 547 |
| 19 | 2.55 | 68 | 91 | 534 |

1. A carbonylation process for preparing an alkanoic acid ester comprising reacting:
(a) an alkene;
(b) a source of Pd;
(c) a bidentate phosphine ligand of formula I;
R¹R²P—R³—R—R⁴—PR⁵R⁶ (I)
wherein P represents a phosphorus atom; R¹, R², R⁵ and R⁶ can independently represent the same or different optionally substituted organic groups containing a tertiary carbon atom through which the group is linked to the phosphorus atom; R³ and R⁴ independently represent optionally substituted lower alkylene groups and R represents an optionally substituted aromatic group;
(d) a source of anions derived from an acid with a pKa<3;
(e) carbon monoxide; and
(f) an alkanol;
under conditions wherein an alkanoic acid ester is produced, wherein said process is performed in the presence of from 0.1 to 3% wt water.

2. The process according to claim 1, which is performed in the presence of from 0.15 to 1.5% wt water.

3. The process according to claim 1, wherein the alkene comprises methyl 2-pentenoate.

4. The process according to any one of claim 1, in which R¹, R², R⁵, and R⁶ are tert-butyl, R³ and R⁴ are methylene, and R is ortho-phenylene.

5. The process according to claim 1, wherein the source of Pd is at least one selected from the group consisting of a palladium halide, palladium carboxylate and Pd₂(dba)₃.

6. The process according to claim 1, wherein the alkanoic acid ester is an ester of formula II,
XOOC—(CH₂)₄—COOY (II)
wherein X and Y are independently a lower alkyl group and/or H.

7. The process according to claim 1, wherein the alkene is a mixture comprising cis- and/or trans-methyl 2-pentenoate, cis- and/or trans- methyl 3-pentenoate, and/or methyl-4-pentenoate.

8. The process according to claim 1, wherein the alkene is ethene.

9. The process according to claim 1, wherein the alkanol is methanol.

10. The process according to claim 1, wherein the source of anions derived from an acid having a pKa below 3.0 is methylsulphonic acid, tert-butyl sulphonic acid and/or 2,4,6-trimethylbenzenesulphonic acid.

11. A process to produce adipic acid dimethyl ester, said process comprising:
a. converting valerolactone into a methyl pentenoate by treatment with methanol, in the presence of an acidic or basic catalyst in gas phase and/or in liquid phase; and
b. converting the methyl pentenoate produced in (a) to adipic acid dimethyl ester in a carbonylation process according to claim 1, wherein the alkanol is methanol.

12. The process according to claim 11, wherein valerolacton is prepared by converting levulinic acid to valerolactone in a hydrogenation reaction.

13. The process according to claim 12, wherein levulinic acid is prepared by converting a C6 carbohydrate to levulinic acid in a hydrolysis reaction.

14. The process according to claim 1, wherein adipic acid dimethyl ester is converted to adipic acid in a hydrolysis reaction.

15. The process according to claim 14, wherein adipate is converted to ammonium adipate using ammonia.

16. The process according to claim 15, wherein ammonium adipate is converted to adiponitril in a dehydration reaction.

17. The process according to claim 16, wherein adiponitril is converted to hexamethylenediamine in a reduction reaction.
